(19) United States
(12) Patent Application Publication
(10) Pub. No.: US 2013/0316453 A1
(43) Pub. Date: Nov. 28, 2013

(54) Composition for Enhancing Cellular Uptake of Carrier Particles and Method for the Same

(71) Applicant: CHANG GUNG UNIVERSITY, Tao-Yuan (TW)

(72) Inventors: Yunn-Hwa MA, Tao-Yuan (TW); Yunn-Lii LEU, Tao-Yuan (TW); Jender WU, Taipei City (TW); Kuo-Chen WEI, Taoyuan County (TW); Yi-Ching LU, Tao-Yuan (TW)

(73) Assignee: CHANG GUNG UNIVERSITY, Tao-Yuan (TW)

(21) Appl. No.: 13/899,338

(22) Filed: May 21, 2013

(30) Foreign Application Priority Data
May 22, 2012 (TW) 101118169

(51) Int. Cl. C12N 5/071

(52) U.S. Cl. CPC C12N 5/0602
USPC 435/375

(57) ABSTRACT

A composition for enhancing cellular uptake of carrier particles comprises a delivery system for a drug or biochemical molecule; and a polyphenolic compound, wherein the polyphenolic compound is added to the drug or biochemical molecule delivery system to enhance cellular uptake of drug or biochemical molecules carried by the delivery system. A method for the same is also disclosed, wherein a polyphenolic compound or its derivative is mixed with an existing delivery system for drug or biochemical molecule, and the mixture is used to deliver drug or biochemical molecules into cells or an organism. The method is easy to operate and does not require further chemical reaction in process of the existing delivery system. The delivery system may include a magnetic carrier that can be guided to a specified region by an external magnetic field, consequently increased the amount of the drug or biochemical molecules acting on target cells.

### BACKGROUND OF THE INVENTION

1. Field of the Invention

The present invention relates to a composition for enhancing cellular uptake of carrier particles and a method for the same, particularly to a composition and method, which use a polyphenol or a derivative thereof to promote the efficiency of a drug or biochemical molecule delivery system and enhance cellular uptake of particles carrying the drug or biochemical molecules.

2. Description of the Related Art

To enhance cellular uptake of drugs or carriers is critical for the drug to reach its intracellular target and exerts therapeutic effects in a target delivery system. The approaches thereof are normally focused on modifying the carriers, including the following technologies: (1) varying the functional groups of the polymers coating the carriers; (2) attaching a specified molecule, such as an antibody or ligand, on the surface of the carrier to generate a specific binding; (3) using a physical agent, such as electric pulse, to increase permeability of cellular membrane. However, the above-mentioned technologies are limited by various factors, such as high technical difficulties, complicated reaction processes, poor efficiency, induction of cellular toxicity or cell death. Besides, the above-mentioned technologies usually fail to achieve the expected cellular uptake efficiency.

There are many polyphenols and their derivatives existing in the nature, such as flavonoids, gallic acids, and catechins. Many of them act as antioxidant and exert several biological effects, such as inhibition of tumor growth, improvement of vascular function, and modulation of the immune system. They have been widely applied to chemical industry, food industry, medical and healthcare industry, etc. Recently, polyphenols and their derivatives have been used as natural food additives to replace synthetic antioxidants and stabilizers.

In the medical field, some polyphenolic derivatives, such as catechins and flavonoids, may interact with cells and influence specific signaling pathways, which may result in hindering angiogenesis, inhibiting tumor growth, or decreasing cholesterol levels. Previous studies indicated that some polyphenolic derivatives, such as gallic acids, exert antibacterial and antiviral effects, and may be used in medicine and healthcare. However, no published document mentioned about applications of polyphenolic derivatives to enhancing cellular uptake of carrier particles. In fact, it is greatly preferable in the related fields to utilize the existing biocompatible materials to improve cellular uptake efficiency of drugs without obviously varying the current medicine fabrication processes.

### SUMMARY OF THE INVENTION

One objective of the present invention is to provide a composition for enhancing cellular uptake of carrier particles, wherein a polyphenol is mixed with a delivery system for a drug or biochemical molecule to enhance cellular uptake of the drug or biochemical molecule.

To achieve the above-mentioned objective, the present invention proposes a composition for enhancing cellular uptake of carrier particles, which comprises a polyphenolic compound and a delivery system for a drug or biochemical molecule.

The present invention also proposes a method for enhancing cellular uptake of carrier particles, which comprises the following steps: mixing a polyphenolic compound with a delivery system for a drug or biochemical molecule; forming a composite with modified surface; and allowing target cells to get in contact with the complex delivery system.

In the above-mentioned composition and method, the polyphenolic compound may be a flavonoid, a derivative of a flavonoid, a gallic acid, or a derivative of a gallic acid. For instance, these compounds may include a flavanone, a flavone, a flavonol, a gallic acid, epigallocatechin (EGC), epigallocatechin gallate (EGCG), methyl gallate, quercetin, a derivative of a flavonoid, or a derivative of a gallic acid.

In the above-mentioned method, a polyphenolic compound is mixed with a delivery system for drug/biochemical molecule via one of the following ways: adding a polyphenol or its derivative to the surface of a drug molecule delivery system; trapping a polyphenol or its derivative inside a drug molecule delivery system; homogeneously mixing a polyphenol or its derivative with a drug molecule delivery system. The drug/biochemical molecule delivery system may be in form of nanoparticles having a diameter of less than 1 μm. In one embodiment, the nanoparticles are magnetic nanoparticles, which can be guided by a magnetic field to the target region.

### BRIEF DESCRIPTION OF THE DRAWINGS

FIG. 1 shows the concentration-dependent effects of gallic acid on cellular uptake of MNP;

FIG. 2 shows the concentration-dependent effects of methyl gallate on cellular uptake of MNP;

FIG. 3 shows the concentration-dependent effects of EGCG on cellular uptake of MNP;

FIG. 4 shows the concentration-dependent effects of ECG on cellular uptake of MNP;

FIG. 5 shows the concentration-dependent effects of quercetin on cellular uptake of MNP; and

FIG. 6 shows that EGCG enhanced cellular uptake of magnetic nanoparticle in a transient and reversible manner.

### DETAILED DESCRIPTION OF THE INVENTION

### Embodiment I

influence of gallic acid on cellular uptake of magnetic nanoparticles (MNP)

Cell culture: Cells were cultured in a growth medium containing 10% fetal bovine serum and antibiotics. The growth medium may be DMEM (Dubelco Modified Eagle Medium) or M199. The antibiotics included penicillin (100 U/ml), streptomycin (100 μ/ml), and amphotericin B (0.25 μg/ml). The cells were cultured in a 37° C. incubator supplied with 5% CO₂. For cellular uptake experiments, the cells were cultured in a 24-well culture plate until 80-90% confluence. Preparation of a gallic acid solution: magnetic nanoparticles (100 μg/ml) and gallic acid (0-20 μM) were added to the growth medium and mixed gently

Cellular uptake of MNPs: The growth medium from the culture plate was replaced with medium containing MNP and gallic acid. The cells were exposure to MNP (100 μg/ml) and gallic acid (0 to 20 μM) in the absence and presence of NdFeB magnet in a 37° C. incubator supplied with 5% CO₂ for 24 hours. Cells were then trpysinized and resuspended in phosphate buffer saline.

Estimation of cellular uptake MNP: The amount of MNP taken up by cells was determined by the potassium thiocyanate (KSCN) assay.

First, the collected cellular pellets were dispersed with a micropipette or a microdismembrator. To decomposed iron oxide (Fe₃O₄) of MNP into ferrous (Fe²⁺) ions and ferric (Fe³⁺) ions, the dispersed cell solutions were treated with 10% (v/v) of hydrochloric acid and incubated at a temperature of 50-60° C. for 4 hours, followed by addition of ammonium persulfate (APS; 1 mg/ml) to oxidize ferrous ions to ferric ions. The

KSCN (1M) was then added to and the mixture, allowing formation of potassium ferricyanide. Amount of cell-associated iron was determined with a plate reader at OD₄₉₀. For calibration, standard curve with known amount of MNP was prepared under identical conditions.

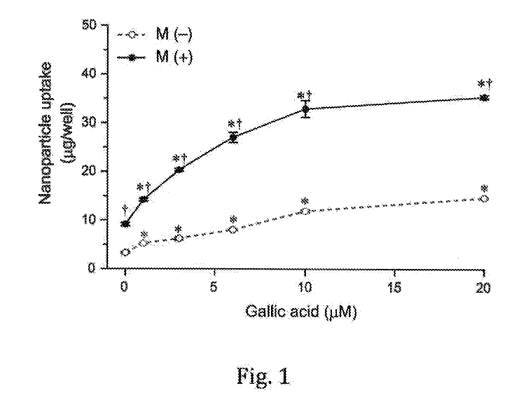

Refer to FIG. 1 showing the influence of gallic acid on cellular uptake of MNP in a concentration-dependent manner, wherein the solid circles denote the case that an external magnetic field is applied underneath while the cells are incubated with MNPs and gallic acid. Meanwhile, the hollow circle denotes the case that cells are incubated with MNPs and gallic acid in the absence of an external magnetic field underneath. From FIG. 1, it is observed that the higher the concentration of gallic acid added in the culture, the greater the amount of MNP taken up by cells. A gallic acid concentration of as low as 1 μM is sufficient to enhance cellular uptake of MNPs. The amount of MNP uptake by the cells increased 50% while incubating in the complex medium containing 1 μM gallic acid both in the absence and presence of a magnetic field underneath in comparison with the cases without gallic acid. In the cases that the cells incubated in the complex medium with 20 μM gallic acid, MNP uptake is even increased by 4 folds. In the present invention, the magnetic field functions to increase nanoparticles contacting cell membranes and provides force to drag magnetic nanoparticles.

### Embodiment II

influence of methyl gallate on cellular uptake of MNP

Embodiment II is basically similar to Embodiment I but different from Embodiment I in that methyl gallate is added to the MNP solution to form a complex medium containing 0-20 μM of methyl gallate.

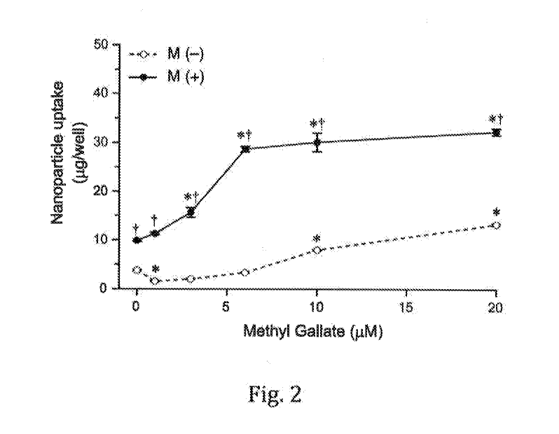

Refer to FIG. 2 showing the concentration-dependent effects of methyl gallate on cellular uptake of MNP, wherein the solid circle denotes the case that an external magnetic field is applied to the incubated cells, and the hollow circle denotes the case that no external magnetic field is applied to the incubated cells. From FIG. 2, it is observed that cellular uptake of MNP begins to reach the plateau at 6 μM of methyl gallate with an external magnetic field, i.e. the effect of methyl gallate on cellular uptake of MNP has reached the maximum. At the concentration of 10 μM, the cellular uptake of MNP is 3 times greater than that without methyl gallate in the presence of an external magnetic field. The effect of methyl gallate on cellular uptake of MNP without an external magnetic field is relatively weaker than that with an external magnetic field. However, the uptake at 10 is still 2 times greater that that at 0 μM in a magnetic field-free environment. It means that methyl gallate can still enhance cellular uptake of MNP in a magnetic field-free environment.

### Embodiment III

influence of EGCG (epigallocatechin gallate) on cellular uptake of MNP

Embodiment III is basically similar to Embodiment I but different from Embodiment I in that EGCG is added to the MNP solution to form a complex medium containing 0-20 μM of EGCG.

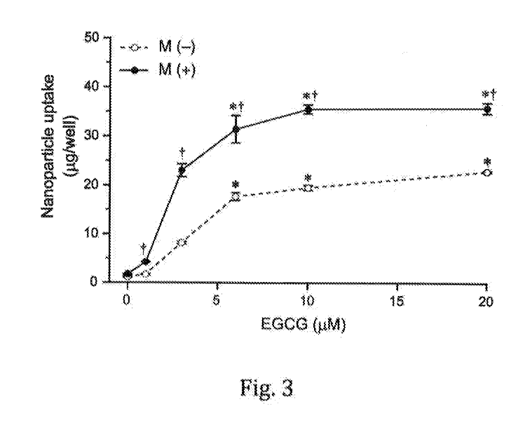

Refer to FIG. 3 showing the concentration-dependent effects of EGCG on cellular uptake of MNP, wherein the solid circle denotes the case that an external magnetic field is applied to the incubated cells, and the hollow circle denotes the case that no external magnetic field is applied to the incubated cells. From FIG. 3, it is observed: the effect of EGCG on cellular uptake of MNP is very obvious. The cellular uptake of MNP was significantly increase by EGCG as low as 3 μM. At 10 μM, EGCG can increase cellular uptake of MNP by 5.7 times in a magnetic field-free environment and by 16 times with an external magnetic field, in comparison with the cases without EGCG.

The enhancement of MNP uptake by EGCG exhibits a concentration-dependent manner in the concentration between 1 to 10 μM. Concentration above 10 μM of EGCG may result in plateau in the cellular uptake of MNP. It is suggested that the effect of EGCG on cellular uptake of MNP has reached the maximum above 10 μM of EGCG.

### Embodiment IV

influence of ECG (epicatechin gallate) on cellular uptake of MNP

Embodiment IV is basically similar to Embodiment I but different from Embodiment I in that ECG is added to the MNP solution to form a complex medium containing 0-20 μM of ECG

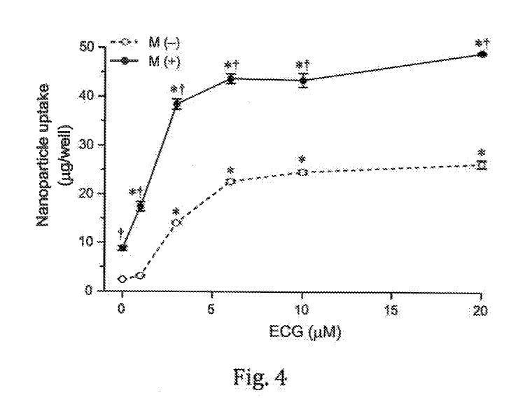

Refer to FIG. 4 showing the concentration-dependent effects of ECG on cellular uptake of MNP. From FIG. 4, it is observed that ECG obviously influences cellular uptake of MNP. Similarly to EGCG, the cellular uptake of MNP was significantly increase by ECG as low as 3 μM. At 10 μM, ECG can increase cellular uptake of MNP by 12 times in a magnetic field-free environment and by 5-6 times with an external magnetic field, in comparison with the cases without ECG.

### Embodiment V

influence of quercetin on cellular uptake of MNP

Embodiment V is basically similar to Embodiment I but different from Embodiment I in that quercetin is added to the MNP solution to form a complex medium containing 0-20 μM of quercetin.

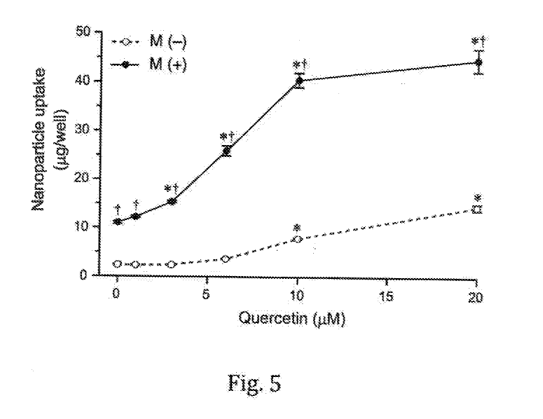

Refer to FIG. 5 showing the concentration-dependent effects of quercetin on cellular uptake of MNP. From FIG. 5, it is observed that the effect of quercetin on cellular uptake of MNP is relative lower that that of gallic acid and its derivatives. In the absence of magnetic field, the cellular uptake of MNP with a high concentration (20 μM) of quercetin is 5 times higher than that without quercetin. There is also a significant increasing in cellular uptake of MNP in a concentration-dependent manner with a magnet underneath. These results indicate that quercetin can also exert an enhance effect in cellular uptake of MNP appropriately.

### Embodiment VI

using EGCG to exemplify the influence of polyphenols and their derivatives on cellular uptake of MNP in different scenarios

There are a assembling of totally 5 groups in the experiments of Embodiment VI, including one control group and 4 experimental groups. In Group 1 (the control group), the system is free from EGCG and incubates with MNPs for 2 hours. In Group 2, the system is reacted with EGCG for 2 hours; next, EGCG is removed from the system; then, the MNPs are reacted with the system for another 2 hours. In Group 3, the system is reacted with EGCG and MNPs for 2 hours. In Group 4, the system is reacted with EGCG for 2 hours; then, the system is reacted wit MNP with EGCG remaining for another 2 hours. In Group 5, the system is reacted with EGCG for 4 hours; then, the system is reacted wit MNP with EGCG remaining for another 2 hours. The experiments are undertaken to evaluate the influence of EGCG existence on cellular uptake of MNP.

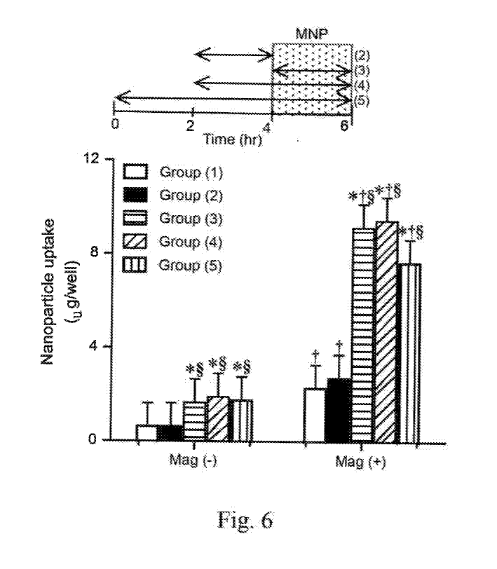

Refer to FIG. 6 showing the EGCG enhanced cellular uptake of MNP in a transient and reversible manner. FIG. 6 shows that no significant enhancement of MNP uptake was observed when cells were pre-exposed to EGCG followed by removal of EGCG prior to a 2 hour-incubation with MNP in Group 2. In the other experimental groups wherein EGCG persistently remains, EGCG works effectively to enhance cellular uptake of MNP. In addition, prolonged incubation with EGCG from 2 to 6 hours does not further enhance amount of cellular MNP with or without magnetic influence. The experiments indicate that the enhancement effect of EGCG requires co-incubation of EGCG and MNPs.

In conclusion, polyphenols and their derivatives can act as assistance in the celluar uptake of extracellular particular materials. When applied to a delivery system for drug/biochemical molecule, polyphenols and their derivatives can thus enhance cellular uptake of the drug or biochemical molecule. In one embodiment, the delivery system for drug/biochemical is realized by magnetic nanoparticles, whereby the molecules of a drug or biochemical molecule can be guided by an external magnetic field to a specified region, wherefore the effect of the drug or biochemical molecule is greatly enhanced.

Further, polyphenols and their derivatives are not necessarily bound to the surface of carriers or trapped inside carriers in their applications. Polyphenols and their derivatives may be mixed with a delivery system for drug/biochemical molecule to form a suspension liquid, and the suspension liquid is then used to deliver a drug or biochemical molecule to the target cells, whereby polyphenols and their derivatives can affect to enhance cellular uptake of the drug or biochemical molecule too. Besides, the method of the present invention need not change the operation way of the existing delivery system for drug/biochemical. In other words, the method of the present invention would not greatly vary the existed fabrication process of the delivery system for drug/biochemical molecule. Therefore, the present invention has high industrial utility, and the application thereof can be utilized instantly.

What is claimed is:

1. A composition for enhancing cellular uptake of carrier particles, comprising:
a delivery system At 10 μM, for a drug or biochemical molecule including at least one biocompatible carrier; and
a polyphenolic compound, wherein the polyphenolic compound is added to the delivery system for the drug or biochemical molecule to enhance cellular uptake of drug or biochemical molecules carried by the delivery system for the drug or biochemical molecule.

2. The composition according to claim 1, wherein the biocompatible carrier is a nanoparticle.

3. The composition according to claim 2, wherein the nanoparticle has a diameter of less than 1 μm.

4. The composition according to claim 2, wherein the nanoparticle is a magnetic nanoparticle.

5. The composition according to claim 1, wherein the polyphenolic compound has a concentration of 1-20 μM.

6. The composition according to claim 1, wherein the polyphenolic compound is selected from a group consisting of flavonoids, derivatives of flavonoids, gallic acids, and derivatives of gallic acids.

7. The composition according to claim 6, wherein the polyphenolic compound is selected from a group consisting of flavanones, flavones, flavonols, gallic acids, EGC (epigallocatechin), EGCG (epigallocatechin gallate), methyl gallate, quercetin, derivatives of flavonoids, and derivatives of gallic acids.

8. The composition according to claim 1, wherein the polyphenolic compound is bound to or trapped inside the delivery system for the drug or biochemical molecule to create a complex system, or wherein the polyphenolic compound is mixed with the delivery system for the drug or biochemical molecule to create a complex system in form of a suspension liquid.

9. A method for enhancing cellular uptake of carrier particles, comprising steps:
using a polyphenolic compound to preparing a polyphenolic solution having a concentration of 1-20 μM;
providing a delivery system for a drug or biochemical molecule delivery system including at least one biocompatible carrier;
combining the polyphenolic solution and the delivery system for the drug or biochemical molecule to form a complex molecule delivery system; and
using the complex molecule delivery system to deliver molecules of a drug or a biochemical to target cells and letting the polyphenolic compound contact the target cells to enhance cellular uptake of molecules of the drug or the biochemical molecule.

10. The method according to claim 9, wherein the polyphenolic compound is bound to or trapped inside the drug or biochemical molecule delivery system to create a complex system, or wherein the polyphenolic compound is mixed with the delivery system for the drug or biochemical molecule to create the complex system in form of a suspension liquid.

11. The method according to claim 9, wherein the biocompatible carrier is a nanoparticle.

12. The method according to claim 11, wherein the nanoparticle is a magnetic nanoparticle.

13. The method according to claim 11, wherein the polyphenolic compound is selected from a group consisting of flavonoids, derivatives of flavonoids, gallic acids, and derivatives of gallic acids.

14. The method according to claim 13, wherein the polyphenolic compound is selected from a group consisting of flavanones, flavones, flavonols, gallic acids, EGC (epigallocatechin), EGCG (epigallocatechin gallate), methyl gallate, quercetin, derivatives of flavonoids, and derivatives of gallic acids.
